(19) United States
(12) Patent Application Publication
(10) Pub. No.: US 2015/0096556 A1
(43) Pub. Date: Apr. 9, 2015

(54) STEERABLE ENDOTRACHEAL INTUBATION APPARATUS, ENDOTRACHEAL INTUBATION COMPONENT SYSTEM AND ENDOTRACHEAL TUBE OF SAME

(71) Applicant: Robert S. Marks, Austin, TX (US)

(72) Inventor: Robert S. Marks, Austin, TX (US)

(21) Appl. No.: 14/046,994

(22) Filed: Oct. 6, 2013

(51) Int. Cl. A61M 16/04

(52) U.S. Cl. CPC A61M 16/0488
USPC 128/200.26; 128/207.14

(57) ABSTRACT

An endotracheal intubation apparatus provides for controlled articulation of tip and central portions of a stylet having an endotracheal tube specifically configured for use therewith mounted thereon. The endotracheal intubation apparatus includes a stylet with tip and central portions that each have a curvature that is selectively and independently adjustable through one or more controls at a handle of the stylet. Through adjustment of the curvature of the tip and central portions of the stylet, a curvature of overlying portions of an endotracheal tube mounted on the stylet can be selectively and independently adjusted. Advantageously, such selectively and independently adjustment of the curvature of the tip and central portions of the stylet allows a contour of the endotracheal tube to be selectively adjusted for accommodating a particular airway anatomy of a patient without requiring removal of the endotracheal intubation apparatus.

### FIELD OF THE DISCLOSURE

The disclosures made herein relate generally to endotracheal tubes and their method of placement and, more particularly, to a steerable endotracheal intubation apparatus, endotracheal intubation apparatus and endotracheal tube of same.

### BACKGROUND

Medical professionals such as, for example, anesthesiologists, emergency responders and clinicians have one common denominator, which is the need to intubate a patient. Intubation is the placement of a tube into a patient's airway in order to allow for gas exchange into the lungs, such as the placement of a patient onto a ventilator. Placing an endotracheal tube is frequently done without difficulty. However, at times, there are more challenging cases and a key consideration in these cases is that failure to properly place the endotracheal tube in a timely manner can yield catastrophic results to the patient.

A device such as, for example, a laryngoscope, video-glidescope or the like is used to allow the medical professional to visualize the anatomy of an airway of a patient. The endotracheal tube remains in the patient after the process is completed for allowing airflow through the patient's respiratory system. A traditional stylet (e.g., prior art) is a manually malleable piece of metal that slides into the endotracheal tube (i.e. the endotracheal intubation apparatus) to allow the endotracheal tube to be maintained in the bend contour (i.e., curvature) of the stylet to assist in placing the endotracheal tube thereby matching the contour of the endotracheal tube to that of the anatomy of the airway of the patient.

Intubation is relatively simplistic in terms of what is needed to place the endotracheal tube in a patient. The process of intubation typically involves using a device to visualize the back of the patient's throat and the airway in order to find the place to put the endotracheal tube. Once the anatomy is seen, the endotracheal intubation apparatus is placed, and the stylet is then removed leaving the endotracheal tube in place. One known device for visualizing the back of the throat is a laryngoscope, which is a special curved piece of metal with a flashlight, which is used to retract the necessary anatomy and illuminate the airway. In some cases, the laryngoscope includes a camera at the tip (i.e., a glidescope) so the medical professional can watch the endotracheal tube placement process on a screen rather than needing to look directly into the patient's mouth. Fiberoptic intubation devices (e.g., a bronchoscope) can also be used in combination with a stylet, which allow for the camera to be placed directly into the endotracheal tube thereby allowing the process of intubation to be performed via direct visualization of the path of the endotracheal tube. In this case, once the target is visualized, the endotracheal tube is slid off of the fiberoptic intubation device once it is in its target location.

A common occurrence when attempting to place an endotracheal tube is the need to pull the entire endotracheal intubation apparatus out of the patient's body and bend the stylet in order to achieve a more optimal angle so that the tip of the endotracheal tube can properly reach the entry point of the larynx. Oftentimes, the stylet needs to be bent in a certain way, and it can be difficult or impossible to know the optimal bend prior to visualizing and/or experiencing the anatomy. For example, sometimes a central portion of the stylet needs to be curved significantly and, depending on the anatomy, the tip sometimes needs to be curved differently.

Removing the endotracheal intubation apparatus (i.e., stylet with mounted endotracheal tube) and needing to change its contour through manual bending of the stylet requires time, which puts the patient at risk of further deoxygenation secondary to a lack of oxygen during the process. Furthermore, changing the contour of the endotracheal intubation apparatus often requires the medical professional to use both hands and, thus, requires them to let go of any device they are using to separately visualize the patient's airway anatomy. The time taken with this process is invaluable and can lead to an adverse situation associated with the patient's oxygen level declining. In addition to the patient's oxygen level declining, there can also be other adverse situations that take place such as, for example, the patient's airway anatomy being distorted from bleeding from the initial attempts secondary to trauma to the airway.

Therefore, an endotracheal intubation apparatus that provides for controlled articulation of tip and central portions of the stylet and an endotracheal tube specifically configured for use therewith would be advantageous, desirable and useful.

### SUMMARY OF THE DISCLOSURE

Embodiments of the present invention are directed to an endotracheal intubation apparatus that provides for controlled articulation of tip and central segments (i.e., portions) of the stylet and an endotracheal tube specifically configured for use therewith. More specifically, embodiments of the present invention are directed to an endotracheal intubation apparatus including a stylet with tip and central segments having a curvature that can be selectively and independently adjustable through one or more controls at a handle of the stylet. Through adjustment of the curvature of the tip and central segments of the stylet, a curvature of overlying portions of an endotracheal tube mounted on the stylet can be selectively and independently adjusted. Advantageously, such selectively and independently adjustment of the curvature of the tip and central portions of the stylet allows a contour of the endotracheal tube to be selectively adjusted for accommodating a particular airway anatomy of a patient without requiring removal of the endotracheal intubation apparatus.

In one embodiment of the present invention, an endotracheal intubation tube comprises a main body having an end segment, a base segment and a middle segment between the end and base segments. A central passage extends through the end segment, the base segment and the middle segment. The end segment has a unit length flexural stiffness substantially less than a unit length flexural stiffness of the middle segment. A length of the end segment is substantially less than a length of the middle segment.

In another embodiment of the present invention, an endotracheal intubation apparatus comprises an endotracheal tube and an endotracheal tube stylet. The endotracheal tube includes a main body having an end segment, a base segment and a middle segment between the end and base segments. A central passage extends through the end segment, the base segment and the middle segment. The endotracheal tube stylet includes a hand-gripping portion and a multi-segment extension portion. The multi-segment extension portion is attached to the hand-gripping portion. The multi-segment extension portion includes a tip segment and a central segment connected to the tip segment. The hand-gripping portion includes at least one curvature adjuster coupled to the tip and central segments for enabling a curvature of each one of the segments to be independently and selectively adjusted. The multi-segment extension portion is positioned within the central passage of the endotracheal tube. The tip segment extends along at least a portion of the length of the end segment of the endotracheal tube. The central segment extends along at least a portion of the length of the middle segment of the endotracheal tube.

In another embodiment of the present invention, an endotracheal intubation component system comprises an endotracheal tube and an endotracheal tube stylet. The endotracheal tube includes a main body having an end segment, a base segment and a middle segment between the first and base segments. A central passage extends through the end segment, the base segment and the middle segment. The endotracheal tube stylet including a hand-gripping portion and a multi-segment extension portion. The multi-segment extension portion is attached to the hand-gripping portion. The multi-segment extension portion includes a tip segment and a central segment connected to the tip segment. The hand-gripping portion includes at least one curvature adjuster coupled to the tip and central segments for enabling a curvature of each one of the segments to be independently and selectively adjusted. The multi-segment extension portion and the central passage of the endotracheal tube are jointly configured for enabling the tip and central segments of the endotracheal tube stylet to be positioned within the central passage of the endotracheal tube such that the tip segment extends along at least a portion of a length of the end segment of the endotracheal tube and the central segment extends along at least a portion of a length of the middle segment of the endotracheal tube.

An endotracheal tube stylet comprises a hand-gripping portion and a multi-segment extension portion. The multi-segment extension portion is attached to the hand-gripping portion. The multi-segment extension portion includes a tip segment and a central segment connected to the tip segment. Both the tip segment and the central segment are movable. The hand-gripping portion includes a curvature adjusting mechanism that is coupled to the tip and central segments for enabling a curvature of each one of the segments to be selectively adjusted.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a side view of an endotracheal tube configured in accordance with an embodiment of the present invention.

FIG. 2 is an end view at a connector portion of the endotracheal tube shown in FIG. 1.

FIG. 3 is a cross-sectional view taken along the line 3-3 in FIG. 1.

FIG. 4 is a cross-sectional view taken along the line 4-4 in FIG. 1.

FIG. 5 is a side view of an endotracheal intubation apparatus configured in accordance with an embodiment of the present invention wherein the endotracheal tube thereof is in a first curvature configuration.

FIG. 6 is a side view of the endotracheal intubation apparatus of FIG. 5 wherein the endotracheal tube thereof is in a second curvature configuration.

FIG. 7 is a side view of the endotracheal intubation apparatus of FIG. 5 wherein the endotracheal tube thereof is in a third curvature configuration.

FIG. 8 is a side view of an endotracheal tube stylet configured in accordance with an embodiment of the present invention.

FIG. 9 is a cross-sectional view taken along the line 9-9 in FIG. 8.

FIG. 10 is a cross-sectional view taken along the line 10-10 in FIG. 8.

### DETAILED DESCRIPTION

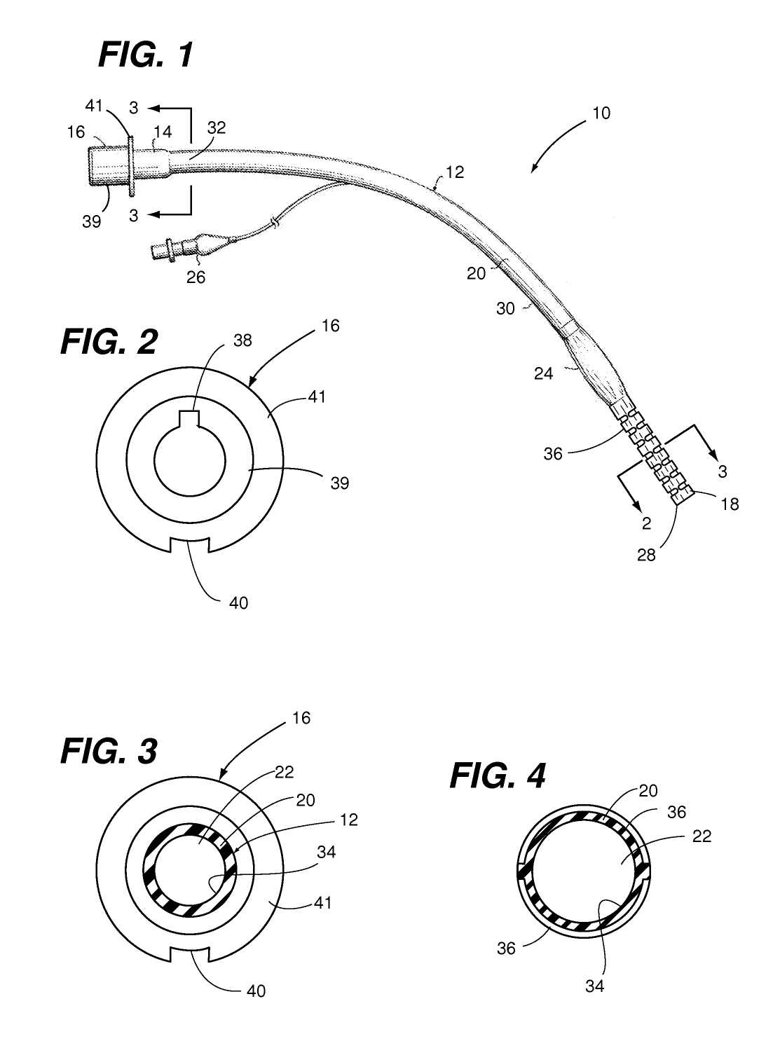

FIGS. 1-4 show various aspects of an endotracheal tube 10 configured in accordance with an embodiment of the present invention. As will be discussed in greater detail herein, the endotracheal tube 10 is specifically configured for use with a stylet configured in accordance an embodiment of the present invention. Jointly, an endotracheal tube and stylet configured in accordance with the present invention jointly define an endotracheal intubation apparatus configured in accordance with an embodiment of the present invention and are components of an endotracheal intubation component system configured in accordance with an embodiment of the present invention.

With reference to FIGS. 1-4, the endotracheal tube 10 is defined by an elongated main body 12 having a proximal end 14 that is attached (e.g., removably) to a connector portion 16, and a distal tip end 18. An inflatable cuff or balloon 24 is provided adjacent distal end 18 and is coupled to an inflation port 26 as is typical for standard, adult endotracheal tubes. For smaller size endotracheal tubes, the cuff 24 and port 26 may be eliminated.

The main body 12 can be formed to have an as-manufactured arcuate shape (e.g., as shown in FIG. 1). The main body 12 includes an end segment 28, a middle segment 30 and a base segment 32. A central passage 22 of the main body 12 (i.e., a gas flow lumen) extends through the end segment 28, the middle segment 30, and the base segment 32. In this regard, the end segment 28, the middle segment 30 and the base segment 32 jointly define an interior surface 34 of the main body 12 that in turn defines the central passage 22 of the main body 12. The central passage 22 provides for flow of gas, such as oxygen, to ventilate a patient. Preferably, but not necessarily, the balloon 24 is not expose to the central passage 22 (e.g., is externally mounted).

The end segment 28 and the middle segment 30 are both flexible for allowing them to be bent into different curvatures (i.e., flexibility provides for selective curvature). Preferably, but not necessarily, the end segment 28 has a unit length flexural stiffness substantially less than a unit length flexural stiffness of the middle segment 30. In one implementation, as shown in FIGS. 1 and 4, the end segment 28 can include one or more recesses 36 in an exterior surface and/or interior surface thereof and a length of the end segment 28 is substantially less than a length of the middle segment 30. The recesses 36 are configured for causing the unit length flexural stiffness of the end segment 28 to be substantially less than the unit length flexural stiffness of the middle segment 30 (e.g., an exterior surface and interior surface of the middle segment 30 is devoid of such recesses). Unit length flexural stiffness refers to a degree of stiffness over a particular unit length (e.g., over 1-inch). Optionally or alternatively, a thickness of at least a portion of the end segment can be substantially less than a thickness of the middle segment thereby causing the unit length flexural stiffness of the end segment to be substantially less than the unit length flexural stiffness of the middle segment. Optionally or alternatively, the end segment 28 can be made from a first polymeric material and the middle segment 30 can be made from a second polymeric material different than the first polymeric material for causing the unit length flexural stiffness of the end segment 28 to be substantially less than the unit length flexural stiffness of the middle segment 30.

As shown in FIGS. 2 and 3, the connector portion 16 of the main body 12 includes a first stylet engagement structure 38 within a ventilator mounting nipple 39 of the connector portion 16 and a second stylet engagement structure 40 within a flange 41 of the connector portion 16. The first stylet engagement structure 38 is a feature (e.g., channel) configured for being engaged by a mating structure of an endotracheal tube stylet for defining a clocked orientation of the endotracheal tube 10 with the endotracheal tube stylet (e.g., defines a relative angular orientation and inhibits unrestricted rotation of the endotracheal tube 10 relative to an attached an engaged endotracheal tube stylet). The second stylet engagement structure 40 is a feature (e.g., slot) configured for being engaged by a mating structure of the endotracheal tube stylet for inhibiting unrestricted translation of the main body 12 with respect to the endotracheal tube stylet, rotation of the main body 12 with respect to the endotracheal tube stylet, or both.

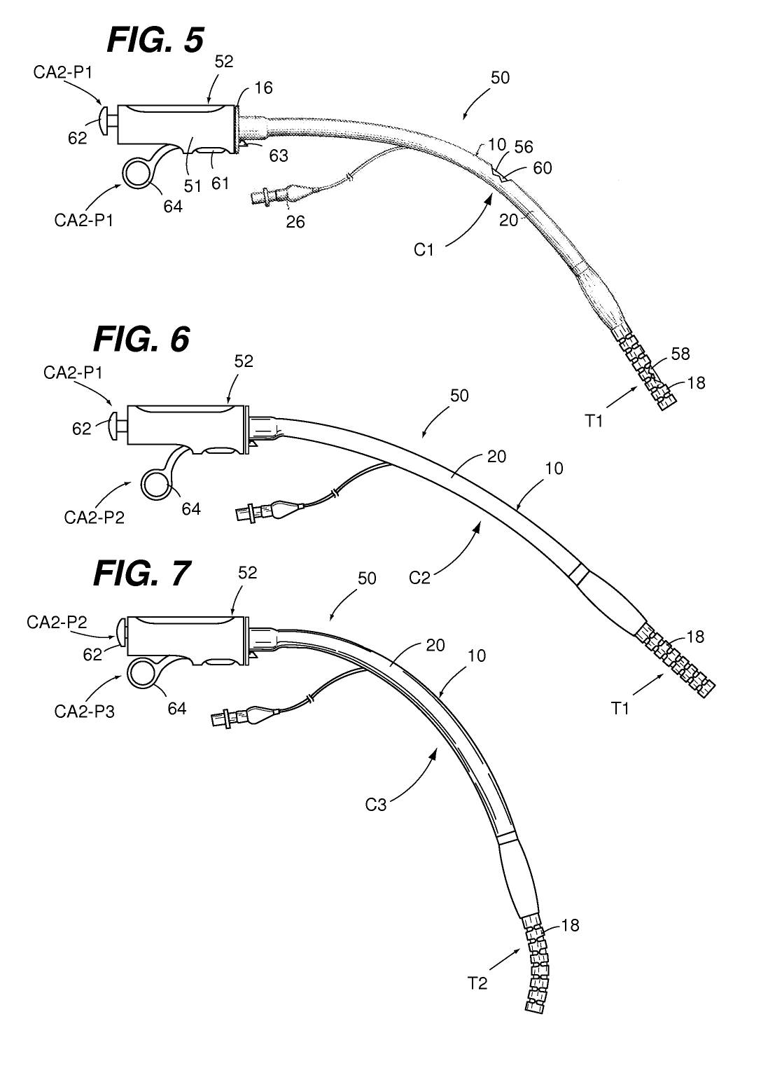

Turning now to FIGS. 5-7, an endotracheal intubation apparatus 50 configured in accordance with an embodiment of the present invention is shown. The endotracheal intubation apparatus 50 includes the endotracheal tube 10 disclosed above in reference to FIGS. 1-4 (i.e., an endotracheal tube configured in accordance with an embodiment of the present invention) and an endotracheal tube stylet 52, which is configured in accordance with an embodiment of the present invention. It is disclosed herein that the endotracheal tube 10 disclosed above in reference to FIGS. 1-4 is one example of an endotracheal tube suitable for use with an endotracheal tube stylet configured in accordance with an embodiment of the present invention. It is disclosed herein that certain standard (e.g., prior art/commercially available) endotracheal tubes may be used in combination with an endotracheal tube stylet configured in accordance with an embodiment of the present invention.

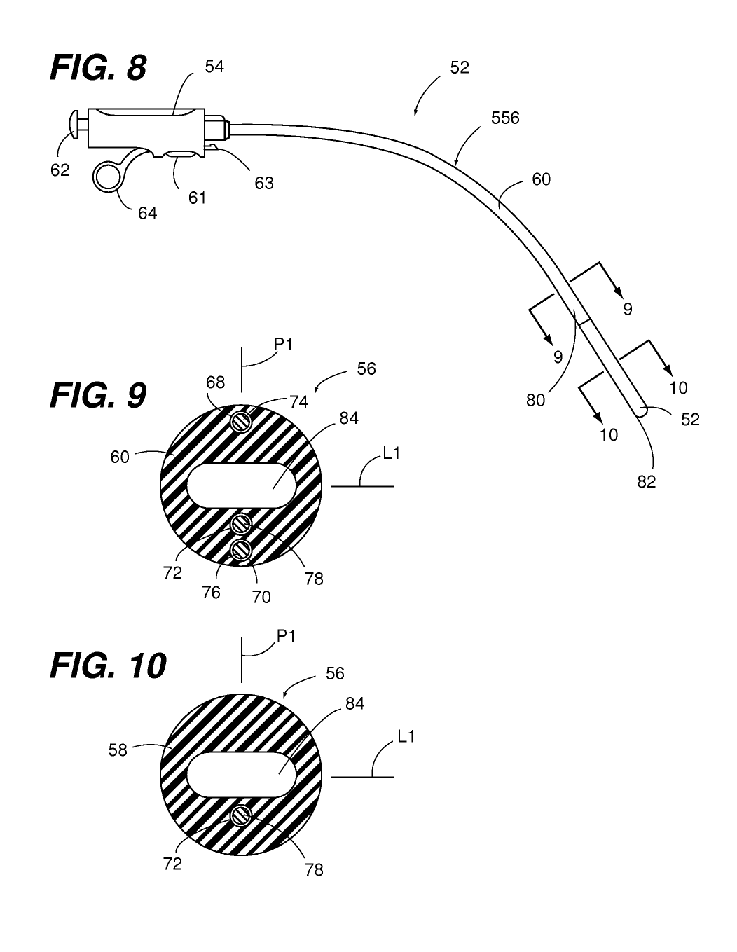

As best shown in FIG. 8, the endotracheal tube stylet 52 includes a hand-gripping portion 54 and a multi-segment extension portion 56. The multi-segment extension portion 56 is attached to the hand-gripping portion 54. The multi-segment extension portion 56 includes a tip segment 58 and a central segment 60 connected to the tip segment 58. The hand-gripping portion 54 includes a tube release mechanism (e.g., button 61) that enables a tube engagement structure 63 to be selectively disengaged from the second stylet engagement structure 40 thereby allowing the endotracheal tube 10 to be detached from the endotracheal tube stylet 52. A mounting portion 65 of the endotracheal tube stylet 52 is configured for having the connector portion 16 of the endotracheal tube 10 mounted thereon. In certain embodiments, the tube release mechanism (e.g., a structure connected to the button 61) can be configured for urging the connector portion 16 of the endotracheal tube 10 out of engagement with the mounting portion 65. Optionally, the mounting portion 65 can include an alignment feature 67 that engages the first stylet engagement structure 38 of the connector portion 16 of the endotracheal tube 10 for providing a clocked orientation of the endotracheal tube 10 with respect to the endotracheal tube stylet 52. It is disclosed herein that all of the alignment features (e.g., first stylet engagement structure 38, a second stylet engagement structure 40, the tube engagement structure 63, and alignment feature 67) can be omitted without departing from the spirit or advantageous functionality of the present invention. It is also disclosed herein that the tube engagement structure 63 in conjunction with the second stylet engagement structure 40 can solely provide for clocked orientation functionality of the endotracheal tube 10 with respect to the endotracheal tube stylet 52. Furthermore, in view of the disclosures made herein, a skilled person will appreciate that the design of the hand-gripping portion 54 can be implemented in a manner allowing use with an endotracheal tube having a connector 16 that is conventionally configured (e.g., a connector configured in accordance with a prior art endotracheal tube).

Referring back to FIGS. 5-7, the multi-segment extension portion 56 is positioned within the central passage of the endotracheal tube 10 such that the tip segment 58 extends along at least a portion of the length of the end segment 18 of the endotracheal tube 10 and the central segment 60 extends along at least a portion of the length of the middle segment 20 of the endotracheal tube 10. The hand-gripping portion 54 includes a first curvature adjuster 62 and a second curvature adjuster 64. The first curvature adjuster 62 is coupled to the tip segment 58 of the multi-segment extension portion 56 for enabling curvature of the tip segment 58 to be selectively altered through movement of the first curvature adjuster 62. The second curvature adjuster 64 is coupled to the central segment 60 of the multi-segment extension portion 56 for enabling curvature of the central segment 60 to be selectively altered through movement of the second curvature adjuster 64. The first curvature adjuster 62 and the second curvature adjuster 64 are independently operable for enabling curvature of the tip segment 58 and the central segment 60, respectively, to be both independently and simultaneously altered.

As shown in FIG. 5, the tip segment 58 is in a first tip segment orientation T1 when the first curvature adjuster 62 is in a first position CA1-P1 and central segment 60 is in a first central segment orientation C1 when the second curvature adjuster 64 is in a first position CA2-P1. As shown in FIG. 6, the tip segment 58 remains in the first tip segment orientation T1 when the first curvature adjuster 62 is in the first position CA1-P1 and central segment 60 moves to a second central segment orientation C2 when the second curvature adjuster 64 is in a second position CA2-P2. As shown in FIG. 7, the tip segment 58 moves to a second tip segment orientation T2 when the first curvature adjuster 62 is in a second position CA1-P2 and central segment 60 moves to a third central segment orientation C3 when the second curvature adjuster 64 is in a third position CA2-P3. These curvature adjuster positions and the relative orientation of the tip segment 58 and central segment 60 of the multi-segment extension portion 56 are for illustrative purposes in the depicted embodiment. A skilled person will appreciate that different control conditions can be implemented and that other configures of curvature adjusters can be implemented. For example, it is disclosed herein that the first curvature adjuster 62 can be a dual-direction adjuster configured in the same or similar manner as the second curvature adjuster 64 thereby allowing for curvature adjustment of the tip segment 58 in opposing directions from a baseline orientation (e.g., opposite the displaced orientation shown in FIG. 7).

Referring now to FIGS. 9 and 10, implementations of mechanisms for providing selective curvature of the multi-segment extension portion 56 is shown. The middle segment 56 includes a first control wire lumen 68, a second control wire lumen 70 and a third control wire lumen 72. A first control wire 74 extends through the first control wire lumen 68, a second control wire 76 extends through the second control wire lumen 70 and a third control wire 78 extends through the third control wire lumen 72. The first and second control wires 74, 76 are each connected to a distal end portion 80 (FIG. 8) of the central segment 60 and to the second curvature adjuster 64 for enabling curvature adjustment of the central segment 60. The third control wire 78 is connected to a distal end portion 82 (FIG. 8) of the tip segment 58 and to the first curvature adjuster 62 for enabling curvature adjustment of the tip segment 58. In the case of the first curvature adjuster 62 being configured for dual-direction adjustment of the tip segment 58, a fourth control wire lumen having a fourth control wire therein can be implemented in conjunction with the third control wire lumen 72 and the third control wire 78 (e.g., adjacent to the first control wire lumen 68 along a first plane P1) for enabling such dual-direction adjustment of the tip segment 58.

The tip segment 58 and the central segment 60 of the multi-segment extension portion 56 each include a central passage 84 having an oblong cross-sectional profile. The oblong cross-sectional profile of the central passage 84 serves to define a preferential direction of curvature of the tip segment 58 and the central segment 60. Specifically, a long axis L1 of the central passage 84 causes the tip segment 58 and the central segment 60 to each preferentially bend within the common plane P1. In this regard, preferably, the tip segment 58 and the central segment 60 each include a structure that limits their flexure only within the common plane P1. In this regard, the curvature adjusters 62, 64, the control wire lumens 68, 70, 72, the control wires 74, 76, 78, and the oblong shaped central passage 84 jointly define a curvature adjusting mechanism. It is disclosed herein that a curvature adjusting mechanism configured in accordance with the present invention is limited to enabling the curvature of the tip and central segments of a multi-segment extension portion of an endotracheal tube stylet configured in accordance with an embodiment of the present invention to be independently and selectively adjusted (i.e., flexed) only within a common plane. Preferably, such adjustment would be inhibited outside of the common plane. In view of the disclosures made herein, a skilled person will appreciate various implantations of a curvature adjusting mechanism for enabling selective and independent control of a tip and central segments of a multi-segment extension portion of an endotracheal tube stylet. For example, U.S. Pat. Nos. 7,008,401; 6,783,510; 6,610,058; and 5,254,088 each disclose subject matter that a skilled person privy to the disclosures made herein would consider (e.g., jointly consider) for implementing one or more suitable approaches for enabling selective and independent control of a tip and central segments of a multi-segment extension portion of an endotracheal tube stylet.

It is disclosed herein that endotracheal tubes in the context of the present invention can be of different lengths. To this end, it is desirable for an endotracheal tube stylet configured in accordance with the present invention to accommodate such different length endotracheal tubes. For example, the mounting portion 65 of the endotracheal tube stylet 52 can be adjustable along a length of the multi-segment extension portion 56. In another example, interposers of different length can be provided for being engaged between the mounting portion 65 of the endotracheal tube stylet 52 and the connector portion 16 of the endotracheal tube 10. Accordingly, in view of the disclosures made herein, a skilled person will appreciate various of approaches for enabling the use of different length endotracheal tubes with an endotracheal tube stylet configured in accordance with the present invention.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in all its aspects. Although the invention has been described with reference to particular means, materials and embodiments, the invention is not intended to be limited to the particulars disclosed; rather, the invention extends to all functionally equivalent technologies, structures, methods and uses such as are within the scope of the appended claims.

What is claimed is:

1. An endotracheal intubation tube, comprising:
a main body having an end segment, a base segment and a middle segment between the first and base segments;
wherein a central passage extends through the end segment, the base segment and the middle segment;
wherein the end segment has a unit length flexural stiffness substantially less than a unit length flexural stiffness of the middle segment; and
wherein a length of the end segment is substantially less than a length of the middle segment.

2. The endotracheal intubation tube of claim 1 wherein at least one of:
a thickness of at least a portion of the end segment is substantially less than a thickness of the middle segment thereby causing the unit length flexural stiffness of the end segment to be substantially less than the unit length flexural stiffness of the middle segment; and
the end segment is made from a polymeric material and the middle segment is made from a second polymeric material for enabling the unit length flexural stiffness of the end segment to be substantially less than the unit length flexural stiffness of the middle segment.

3. The endotracheal intubation tube of claim 1 wherein at least one of an exterior surface of the main body at the end portion and an interior surface of the main body within the central passage thereof at the end portion thereof includes one or more recesses therein that are configured for causing the unit length flexural stiffness of the end segment to be substantially less than the unit length flexural stiffness of the middle segment.

4. The endotracheal intubation tube of claim 1 wherein the main body includes a stylet engagement structure at the base segment thereof for enabling the main body to be engaged with a stylet in a manner that inhibits at least one of unrestricted translation of the main body with respect to the stylet and rotation of the main body with respect to the stylet.

5. The endotracheal intubation tube of claim 4 wherein at least one of:
a thickness of at least a portion of the end segment is substantially less than a thickness of the middle segment thereby causing the unit length flexural stiffness of the end segment to be substantially less than the unit length flexural stiffness of the middle segment; and
the end segment is made from a first polymeric material and the middle segment is made from a second polymeric material different than the first polymeric material for causing the unit length flexural stiffness of the end segment to be substantially less than the unit length flexural stiffness of the middle segment.

6. The endotracheal intubation tube of claim 4 wherein at least one of an exterior surface of the main body at the end portion and an interior surface of the main body within the central passage thereof at the end portion thereof includes one or more recesses therein that are configured for causing the unit length flexural stiffness of the end segment to be substantially less than the unit length flexural stiffness of the middle segment.

7. An endotracheal intubation apparatus, comprising:
an endotracheal tube including a main body having an end segment, a base segment and a middle segment between the end and base segments, wherein a central passage extends through the end segment, the base segment and the middle segment; and
an endotracheal tube stylet including a hand-gripping portion and a multi-segment extension portion, wherein the multi-segment extension portion is attached to the hand-gripping portion, wherein the multi-segment extension portion includes a tip segment and a central segment connected to the tip segment, wherein the hand gripping portion includes at least one curvature adjuster; wherein the at least one curvature adjuster is coupled to the tip and central segments for enabling a curvature of each one of said segments to be independently and selectively adjusted, wherein the multi-segment extension portion is positioned within the central passage of the endotracheal tube, wherein the tip segment extends along at least a portion of the length of the end segment of the endotracheal tube and wherein the central segment extends along at least a portion of the length of the middle segment of the endotracheal tube.

8. The endotracheal intubation apparatus of claim 7 wherein the tip segment and the central segment are each movable within a common plane and are each substantially inhibited from movement outside of the common plane.

9. The endotracheal intubation apparatus of claim 7 wherein a curvature adjusting mechanism that comprises the at least one curvature adjuster is limited to enabling the curvature of each one of said segments to be independently and selectively adjusted only within a common plane.

10. The endotracheal intubation apparatus of claim 7 wherein:
the main body of the endotracheal tube includes a stylet engagement structure at the base segment thereof;
the endotracheal tube stylet includes a tube engagement structure integral with at least one of the hand-gripping portion and the multi-segment extension portion; and
wherein the stylet engagement structure and the tube engagement structure are jointly configured for being engaged with each other for enabling the multi-segment extension portion to be positioned within the central passage of the endotracheal tube the main body of the endotracheal tube in a manner that inhibits at least one of unrestricted translation of the main body with respect to the multi-segment extension portion and rotation of the main body with respect to the multi-segment extension portion.

11. The endotracheal intubation apparatus of claim 7 wherein the endotracheal tube stylet includes a tube release mechanism for enabling the stylet engagement structure and the tube engagement structure to be selectively disengaged from each other.

12. The endotracheal intubation apparatus of claim 7 wherein at least one of:
a thickness of at least a portion of the end segment is substantially less than a thickness of the middle segment thereby causing the unit length flexural stiffness of the end segment to be substantially less than the unit length flexural stiffness of the middle segment; and
the end segment is made from a first polymeric material and the middle segment is made from a second polymeric material different than the first polymeric material for causing the unit length flexural stiffness of the end segment to be substantially less than the unit length flexural stiffness of the middle segment.

13. The endotracheal intubation apparatus of claim 7 wherein at least one of an exterior surface of the main body at the end portion and an interior surface of the main body within the central passage thereof at the end portion thereof includes one or more recesses therein that are configured for causing the unit length flexural stiffness of the end segment to be substantially less than the unit length flexural stiffness of the middle segment.

14. The endotracheal intubation apparatus of claim 7 wherein:
the hand-gripping portion includes a plurality of curvature adjusters;
a first one of the curvature adjusters is coupled to the tip segment of the multi-segment extension portion for enabling curvature of the tip segment to be selectively altered through movement of the first one of the curvature adjusters;
a second one of the curvature adjusters is coupled to the central segment of the multi-segment extension portion for enabling curvature of the central segment to be selectively altered through movement of the second one of the curvature adjusters; and
the first and second ones of the curvature adjusters are independently operable for enabling curvature of the tip and central segments, respectively, to be both independently and simultaneously altered.

15. The endotracheal intubation apparatus of claim 14 wherein at least one of:
a thickness of at least a portion of the end segment is substantially less than a thickness of the middle segment thereby causing the unit length flexural stiffness of the end segment to be substantially less than the unit length flexural stiffness of the middle segment; and
the end segment is made from a first polymeric material and the middle segment is made from a second polymeric material different than the first polymeric material for causing the unit length flexural stiffness of the end segment to be substantially less than the unit length flexural stiffness of the middle segment.

16. The endotracheal intubation apparatus of claim 14 wherein at least one of an exterior surface of the main body at the end portion and an interior surface of the main body within the central passage thereof at the end portion thereof includes one or more recesses therein that are configured for causing the unit length flexural stiffness of the end segment to be substantially less than the unit length flexural stiffness of the middle segment.

17. An endotracheal intubation component system, comprising:
an endotracheal tube including a main body having an end segment, a base segment and a middle segment between the end and base segments, wherein a central passage extends through the end segment, the base segment and the middle segment; and
an endotracheal tube stylet including a hand-gripping portion and a multi-segment extension portion, wherein the multi-segment extension portion is attached to the hand-gripping portion, wherein the multi-segment extension portion includes a tip segment and a central segment connected to the tip segment, wherein the hand-gripping portion includes at least one curvature adjuster coupled to the tip and central segments for enabling a curvature of each one of said segments to be independently and selectively adjusted, wherein the multi-segment extension portion and the central passage of the endotracheal tube are jointly configured for enabling the tip and central segments of the endotracheal tube stylet to be positioned within the central passage of the endotracheal tube such that the tip segment extends along at least a portion of a length of the end segment of the endotracheal tube and the central segment extends along at least a portion of a length of the middle segment of the endotracheal tube.

18. The endotracheal intubation component system of claim 17 wherein the tip segment and the central segment are each movable within a common plane and are each substantially inhibited from movement outside of the common plane.

19. The endotracheal intubation component system of claim 17 wherein the curvature adjusting mechanism is limited to enabling the curvature of each one of said segments to be independently and selectively adjusted only within a common plane.

20. The endotracheal intubation component system of claim 17 wherein:
the main body of the endotracheal tube includes a stylet engagement structure at the base segment thereof;
the endotracheal tube stylet includes a tube engagement structure integral with at least one of the hand-gripping portion and the multi-segment extension portion; and
wherein the stylet engagement structure and the tube engagement structure are jointly configured for being engaged with each other for enabling the multi-segment extension portion to be positioned within the central passage of the endotracheal tube the main body of the endotracheal tube in a manner that inhibits at least one of unrestricted translation of the main body with respect to the multi-segment extension portion and rotation of the main body with respect to the multi-segment extension portion.

21. The endotracheal intubation component system of claim 17 wherein the endotracheal tube stylet includes a tube release mechanism for enabling the stylet engagement structure and the tube engagement structure to be selectively disengaged from each other.

22. The endotracheal intubation component system of claim 17 wherein at least one of:
a thickness of at least a portion of the end segment is substantially less than a thickness of the middle segment thereby causing the unit length flexural stiffness of the end segment to be substantially less than the unit length flexural stiffness of the middle segment; and
the end segment is made from a first polymeric material and the middle segment is made from a second polymeric material different than the first polymeric material for causing the unit length flexural stiffness of the end segment to be substantially less than the unit length flexural stiffness of the middle segment.

23. An endotracheal tube stylet, comprising:
a hand-gripping portion;
a multi-segment extension portion attached to the hand-gripping portion, wherein the multi-segment extension portion includes a tip segment and a central segment connected to the tip segment and wherein both the tip segment and the central segment are movable; and
a curvature adjusting mechanism coupled to both the tip and central segments for enabling a curvature of each one of said segments to be selectively adjusted.

24. The endotracheal tube stylet of claim 23 wherein:
the curvature adjusting mechanism includes a plurality of curvature adjusters;
a first one of the curvature adjusters is coupled to the tip segment of the multi-segment extension portion for enabling curvature of the tip segment to be selectively altered through movement of the first one of the curvature adjusters;
a second one of the curvature adjusters is coupled to the central segment of the multi-segment extension portion for enabling curvature of the central segment to be selectively altered through movement of the second one of the curvature adjusters; and
the first and second ones of the curvature adjusters are independently operable for enabling curvature of the tip and central segments, respectively, to be both independently and simultaneously altered.

25. The endotracheal tube stylet of claim 24 wherein the tip segment and the central segment are each movable within a common plane and are each substantially inhibited from movement outside of the common plane.

26. The endotracheal tube stylet of claim 23 wherein the curvature adjusting mechanism is limited to enabling the curvature of each one of said segments to be independently and selectively adjusted only within a common plane.

27. The endotracheal tube stylet of claim 24 wherein the endotracheal tube stylet includes a tube engagement structure integral with at least one of the hand-gripping portion and the multi-segment extension portion for enabling a main body of an endotracheal tube to be engaged with the multi-segment extension portion in a manner that inhibits at least one of unrestricted translation of the main body with respect to the multi-segment extension portion and rotation of the main body with respect to the multi-segment extension portion.
